(19) United States
(12) Patent Application Publication
(10) Pub. No.: US 2008/0008761 A1
(43) Pub. Date: Jan. 10, 2008

(54) Pharmaceutical Formulation Administrable Orally In Terms Of A Taste, Consistency And A Dosing

(75) Inventor: Christian Cardon, Brugge (BE)

Correspondence Address: DENNISON, SCHULTZ & MACDONALD, 1727 KING STREET, SUITE 105, ALEXANDRIA, VA, 22314, US

(73) Assignee: CARDON PHAMACEUTICALS NV, Oostkamp (BE)

(21) Appl. No.: 11/722,777

(22) PCT Filed: Jan. 4, 2006
(86) PCT No.: PCT/IB06/00003

§ 371 Date: Jul. 5, 2007

(30) Foreign Application Priority Data
Jan. 7, 2005 (FR) 0500188

(51) Int. Cl. A61K 9/14

(52) U.S. Cl. 424/489

(57) ABSTRACT

A method for preparing a pharmaceutical formulation which is orally administrable in terms of a taste, consistency and a dosing, including the steps of (a) grinding a solid pharmaceutical formulation containing one or several active substances and excipients in such a way that a powder is obtained, and (b) mixing the powder with one or several excipients for transforming the powder into a formulation having a consistency suitable for the oral administration (gel, liquid, paste, . . . ) and with one or several excipients for masking the taste of the solid pharmaceutical formulation to be ground in order to obtain a formulation suitable for the oral administration in terms of taste, consistency and dosing. A device for carrying out the method is also disclosed.

The subject of the present invention is a pharmaceutical formulation suitable for an administration by oral route in terms of taste, consistency and dosage, the preparation method for said pharmaceutical formulation and the device used for the preparation of said formulation.

The problems of acceptance of medicines (particularly in the form of tablets, lozenges, pills, capsules, hard gelatine capsules, etc.) by oral route in mammals are in most cases due to the bad taste of the medicine, the size of the medicine, which is too large, and also the fact that the elderly, children and household pets have difficulties swallowing the medicine.

The difficulty of swallowing a medicine by oral route for the abovementioned reasons is behind the poor follow up by the patient of a treatment prescribed by a physician or a veterinary surgeon, which poses a serious problem. Indeed, not only does the patient not get better but, in addition, stopping the treatment may aggravate his situation.

There also exists a limitation in the use of tablets released on the market when the dosage per tablet is not adapted to the species concerned, for example in the case of household pets. This disadvantage is also bothersome when the dosage of a medicine changes as a function of the evolution of an illness.

In order to improve the tolerance to a treatment, several solutions have been described in the prior art. For instance, the coating around a tablet, for example based on sugar or resin, enables the taste of a medicine to be improved. However, in certain cases, the coating of a tablet only enables tablets of quite large size to be obtained.

It is also possible to form the coating uniquely around the active ingredients. Such a coating will be based for example on gum or resin. In this manner, one manufactures granules or an active ingredient that is ready to be used with the other ingredients (excipients) to form a tablet. The disadvantage is however that one remains with a coating specific to the active ingredient; furthermore, the difficulty of easily swallowing remains due to the size of the tablet.

For certain medicines, a formulation in the form of syrup, paste or drops is available, in order to find a solution to administer medicines such as paracetamol, codeine, vermifuges, haloperidol, etc.

Unfortunately, most medicines released on the market are only available in the form of tablets.

In general, when a medicine cannot be administered by oral route, it is then administered in the form of injection; the disadvantage of this method is that only a health professional can administer it.

A technique (known as pill-popper or pill-gun) also exists in which one uses tongs (of the sugar tong type) to introduce the medicine (for example a full tablet) directly into the throat. However, this comprises the serious risk that the tablet arrives in the respiratory tracts rather than in the digestive tracts.

The inventor of the present invention has now developed a preparation method and a device for implementing said method enabling pharmaceutical formulations particularly well suited to an administration by oral route to be obtained, particularly in terms of consistency (gel, liquid, paste, etc.) and taste (pleasant in the mouth).

Thus, the subject of the present invention is a preparation method for a pharmaceutical formulation suitable for an administration by oral route in terms of taste, consistency and dosage, characterised in that it comprises the following steps:

(a) grinding a solid pharmaceutical formulation, comprising one or several active ingredients and excipients, until a powder is obtained and,

(b) mixing the powder thereby obtained with:

one or several excipients enabling said powder to be transformed into a formulation having a consistency suitable for an administration by oral route (gel, liquid, paste, etc.), and

one or several excipients enabling the taste of the solid pharmaceutical formulation intended to be ground to be masked,

in order to obtain a formulation suitable for an administration by oral route in terms of taste, consistency and dosage.

Solid pharmaceutical formulation is herein taken to mean for example, a tablet, a lozenge, a pill, a capsule, a hard gelatine capsule, etc.

According to one advantageous embodiment of the method of the invention, the powder obtained at the end of the step of grinding of the solid pharmaceutical formulation has a particle size ranging from 60 to 400 microns, and preferably from 100 to 300 microns,

According to an advantageous embodiment of the method of the invention, the excipient(s) enabling the powder obtained at the end of the grinding step to be transformed into a formulation having a consistency suitable for an administration by oral route such as a gel, a liquid or a paste are chosen among the group constituted by resins, polymers soluble or not soluble in water.

As resin, one may for example use Amberlite®, Duolite® or Eudragit®.

By way of examples of polymers soluble in water, one may cite hydroxypropylmethyl cellulose, hydroxypropyl cellulose, polyvinylpyrrolidone and polyvinylic alcohols.

By way of examples of polymers not soluble in water, one may cite methacrylate and aminoalkyl methacrylate copolymers such as the copolymers ethyl acrylate/methyl methacrylate and the copolymers ethyl acrylate/methylmethacrylate/trimethylammonioethylmethacrylate. These are sold under the brand name Eudragit@ (Röhm Pharma).

Polymer not soluble in water is herein taken to mean a polymer insoluble at a concentration of 10 mg per ml of water.

The use of polymers in the preparation of the pharmaceutical formulation of the invention enables a formulation having a viscosity ranging from 50 mPa to 3000 mPa, and preferably from 500 mPa to 1500 mPa, to be obtained.

The quantity of excipients enabling the powder obtained at the end of the grinding step to be transformed into a formulation having a consistency suitable for an administration by oral route, such as for example a paste, varies from 80 to 98% in weight, and preferably from 90 to 96% in weight compared to the total weight of the final oral formulation.

Final oral formulation is herein taken to mean the formulation obtained at the end of the preparation method of the invention.

According to another advantageous embodiment of the method of preparation of the invention, the excipient(s) enabling the taste of the solid pharmaceutical formulation intended to be ground to be masked are chosen among the group constituted by taste enhancers and flavours.

By adding a flavour or an odoriferous product, one obtains a pharmaceutical formulation particularly well accepted by mammals.

By way of example of flavour, one may cite a flavour known as “TCM F53” (TCM Healthcare London Ltd, United Kingdom), of chicken, fish, malt or liver.

The excipients that are responsible for masking the occasional bitter taste of certain medicines are for example polymers that form a “network” around the medicine in powder, thereby preventing direct contact of the medicine with the taste buds of the tongue.

The quantity of excipients enabling the taste of the solid pharmaceutical formulation intended to be ground to be masked varies from 4% to 30% in weight, and preferably from 10% to 20% in weight compared to the total weight of the final, oral formulation.

According to an advantageous embodiment of the method of the invention, it is also possible to add during the mixing step, one or several preservatives such as potassium sorbate, citric acid, sodium benzoate, EDTA salts, a paraben such as methyl, ethyl, propyl and butyl p-hydroxybenzoates.

The method of preparation of the invention is then characterised in that when the quantity of active ingredient of the solid pharmaceutical formulation before grinding varies from 1% to 20% in weight compared to the total weight of the tablet, the quantity of active ingredient in the final oral formulation varies from 0.1% to 5% in weight compared to the total weight of the formulation.

In principle, one cannot administer more than five tablets, because it is then difficult to mask the taste.

A further subject of the present invention is a pharmaceutical formulation suitable for an administration by oral route in terms of taste, consistency and dosage, capable of being obtained according to the method as described above.

A yet further subject of the present invention is a device or apparatus intended for the preparation of a pharmaceutical formulation suitable for an administration by oral route in terms of taste, consistency and dosage, characterised in that it comprises essentially:

an electric motor (2),

a grinder (6) situated around an axle (5),

a mixer (7) situated around the axle (5),

a compartment (8) enabling a solid formulation to be conveyed up to the grinder (6).

The grinder (6) is intended to mill a solid pharmaceutical formulation, for example one or several tablets, until a powder having a particle size ranging from 60 to 400 microns, and preferably from 100 to 300 microns, is obtained.

The mixer (7) enables the powder from the grinding of the oral formulation to be mixed in a homogeneous manner with the excipient(s) used to obtain the desired consistency and taste of the final pharmaceutical formulation, until a formulation suitable for an administration by oral route in terms of consistency (gel, liquid, paste, etc.) and taste is obtained.

The device of the invention is more particularly characterised in that it further comprises:

one or several batteries (1),

one or several pinions (3) and (4),

one or several springs (9) inside the compartment (8),

one or several screws (10) attached to the axle (5) and situated inside the mixer (7).

The electric motor (2) generates the rotation of the pinions (3) and (4), which themselves generate the rotation of the axle (5) and the grinder (6) integral with said axle (5). The spring(s) (9) of the compartment (8) convey a solid formulation intended to be ground up to the grinder (6).

According to an advantageous embodiment, the device of the invention is characterised in that the mixer (7) and compartment (8) are detachable parts of the device.

According to another advantageous embodiment, the device of the invention is characterised in that it further comprises a graduated syringe, which may be sealed.

A yet further subject of the invention is a preparation method for a pharmaceutical formulation suitable for an administration by oral route in terms of taste, consistency and dosage, as described above, characterised in that it comprises the use of the device of the invention as described above.

### DESCRIPTION OF FIGURES

FIGS. 1a and 1b show a cross sectional view of the device of the invention.

FIGS. 2, 3 and 4 show, in three dimensions, a detailed view of different constituent elements of the device of the invention.

FIGS. 5a, 5b and 5c show, under different angles, a three dimensional overall view of the device of the invention.

FIG. 6 shows a view of the syringe.

The following examples illustrate the invention, but do not limit it in any way.

### EXAMPLE 1

### Pharmaceutical Formulation in Liquid Form

One obtains 0.525 ml of a powder by grinding 5 tablets of 150 mg, which is equivalent to a total of 0.75 ml multiplied by the average conversion factor of 0.7).

| Powder | 0.525 ml |
|---|---|
| Amberlite@ 64 (polacrilex resin) | 10% |
| Eudragit@ EPO | 10% |
| {methacrylic copolymer) | |
| Propyleneglycol (moistening agent) | 10% |
| Sorbitol syrup (70%) (support) | 58% |
| Sodium benzoate (preservative) | 1% |
| Glycine (sweetener) | 7% |
| Taste (chicken flavour) | 4% |

By way of example of tablet used, one may cite Prednisolone 5 mg from the Kela laboratories.

### EXAMPLE 2

### Pharmaceutical Formulation in Gel Form

One obtains 0.7 ml of a powder by grinding 5 tablets of 200 mg, which is equivalent to a total of 1 ml multiplied by the average conversion factor of 0.7).

| Powder | 0.7 ml |
|---|---|
| Sodium carboxymethylcellulose (thickener) | 2% |
| Propyleneglycol | 3% |
| Potassium sorbate (preservative) | 1.2% |
| Sorbitol syrup (70%) | 68.8% |
| Duolite ® AP 143 | 5% |
| (styrene/divinylbenzene polymer) | |
| NaOH buffer pH 9 | 17% |
| Taste (fish flavour) | 3% |

### EXAMPLE 3

### Preparation of Excipients of the Formulation Described in the Example 2 Above

The different excipients that will be mixed with the powder of the tablet are prepared according to the desired quantities and are firstly mixed with each other in the order indicated hereafter.

The sodium carboxymethylcellulose is moistened by the propyleneglycol and one adds to the mixture thereby obtained Duolite AP 143.

The fish flavour is then added, then the potassium sorbate (preservative).

The sorbitol syrup is progressively added, firstly in small quantities, then more and more, in order to obtain the necessary volume and for it to be homogeneous.

The buffer is progressively added in small quantities, until the pH is at a value of 9.

### EXAMPLE 4

### Pharmaceutical Formulation in Gel Form

Tablets to be ground: 3.3%

Propylene glycol: 10%

Sodium methylcellulose: 2%

Duolite®: 5%

Fish flavour: 5%

Potassium sorbate: 1%

Sorbitol syrup: 63.7%

NaOH buffer: 10%

### EXAMPLE 5

### Embodiment of the Device of the Invention

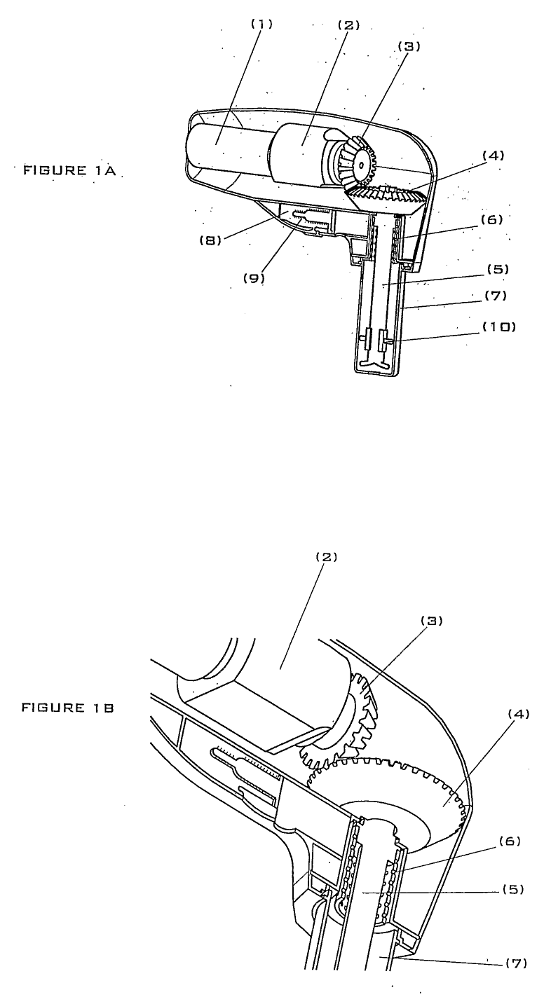

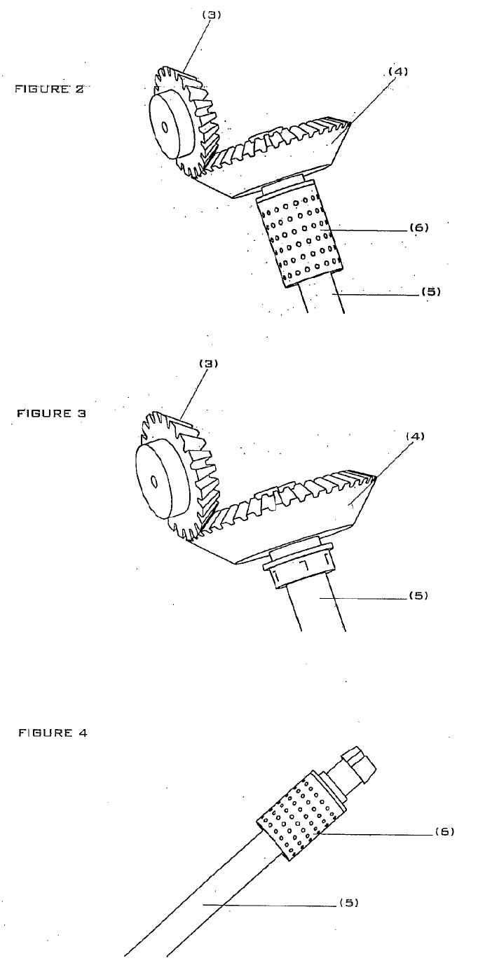

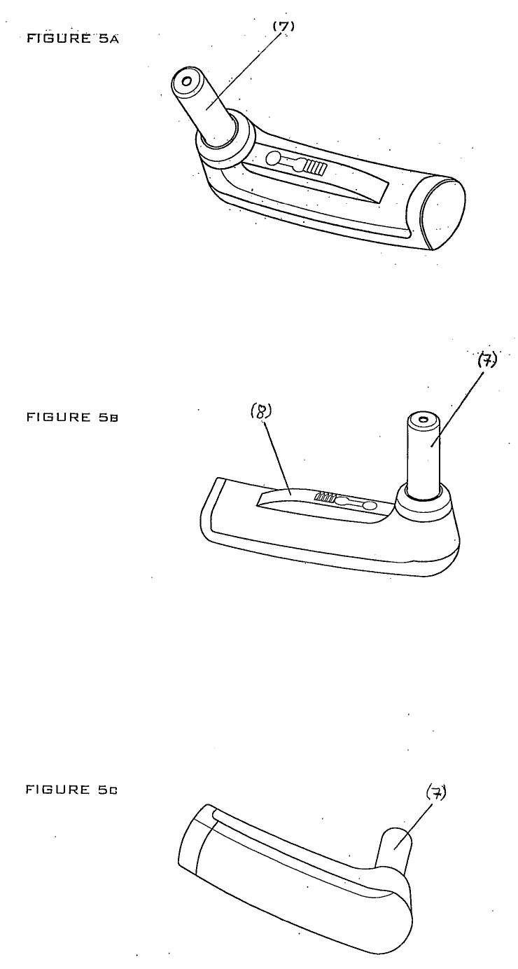

The device according to the invention, shown schematically in FIGS. 1 to 5, comprises more specifically:

one or several batteries (1),

an electric motor (2) generating the rotation of the pinions (3) and (4),

an axle (5) driven by said pinions (3) and (4), and driving the rotation of the grinder (6),

a grinder (6) intended to grind a solid formulation such as for example a tablet, situated around the axle (5),

a compartment (8) intended to receive the solid formulation to be ground, comprising one or several springs (9) enabling said formulation to be conveyed up to the grinder (6),

a mixer (7) situated around the axle (5), equipped with one or several screws (10) attached to the axle (5).

According to an advantageous embodiment, it is for example possible to use two cylindrically shaped batteries, of 1.5 V power. The pinions (3) and (4), activated by the electric motor (2), have a rotation that can reach 5000 rpm. Said pinions (3) and (4) are speed reducers, in order to increase the power of the motor (2).

According to another advantageous embodiment, the grinder (6) is of cylindrical shape with a diameter of around 1 cm and a thickness of 0.3 cm. It is in stainless aluminium, turns with a maximum speed of 15000 rpm and produces a powder with a particle size of 75 microns.

The compartment (8) is detachable in order to assure its maintenance.

The mixer (7) is intended to mix respectively:

(a) the powder of the ground tablet and,

(b) the excipients enabling said powder to be transformed into a formulation having a taste and a consistency suitable for an administration by oral route.

The mixer (7) comprises two screws (10) attached to the axle (5); when the axle is rotating, the screws turn in the opposite direction.

The mixer (7) is detachable, thereby making it possible to clean the device of the invention and thus avoid any interaction with other previously ground tablets.

The assembly may be assembled in a leak tight envelope, a joint in rubber or similar material assuring leak tightness between the mixer (7) and the other elements.

According to another advantageous embodiment of the invention, the device comprises a syringe, independent of or connected to the mixer (7), for example a graduated syringe, enabling all of the excipients to be administered at the desired dosage.

### EXAMPLE 6

### Description of the Syringe

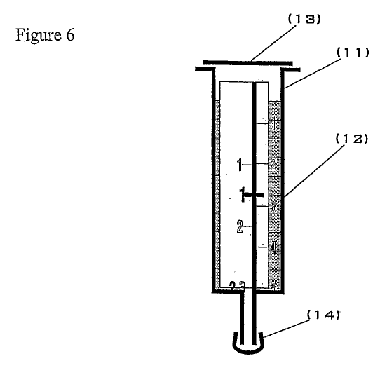

The syringe, shown schematically in FIG. 6, comprises:

a main body (11),

a graduation scale (12),

a leak tight cover (13),

a tapered and sealable end (14).

According to an advantageous embodiment, the main body of the syringe will have a volume of at least 10 ml, a height of 7 cm and a diameter of 1.5 cm. It will be in transparent material to make it possible to view the formation of the mixture. The syringe will be filled with 7.5 ml of the formulation. The average volume released after mixing is 8 ml. This volume varies as a function of the number of tablets, the volume of each tablet, the volume freed by the active agent, the volume freed by the excipients in the tablet.

The main body of the syringe is equipped with a scale, which, according to one embodiment, divides the total volume of the syringe into 5 parts, 4 parts, 3 parts or 2 parts.

The cover (13) is intended in particular to enable the conservation of the formula.

One may append to the syringe a label to note the name of the patient, the date, the name of the medicine, the dosage, the name of the physician or veterinary surgeon.

To use the syringe in combination with the mixer device, after having removed the cover, one fastens said syringe onto the device by screwing it, clipping it or fitting it together in any other equivalent manner.

According to this embodiment, the tablets are ground and the powder is mixed in the mixer with the excipients to obtain a formulation in the form of a powder that falls into the syringe. The formulation is visible in the transparent syringe, for control by the operator. He then removes the syringe and puts on the cover. He puts on the label and places the syringe in a sealed bag until the formulation thereby obtained is used.

According to another embodiment, the syringe is used alone both as means of mixing and dosing. This type of use is possible when the medicine does not need to be ground. After dosing, one proceeds as indicated above.

1. Preparation method for a pharmaceutical formulation suitable for administration by oral route in terms of taste, consistency and dosage, characterised in that it comprises the following steps:
(a) grinding of a solid pharmaceutical formulation, comprising one or several active ingredients and excipients, until a powder is obtained, and
(b) mixing the powder thereby obtained with:
one or several excipients enabling said powder to be transformed into a formulation having a consistency suitable for an administration by oral route (gel, liquid, paste, etc.), and
one or several excipients enabling the taste of the solid pharmaceutical formulation intended to be ground to be masked,
in order to obtain a formulation suitable for an administration by oral route in terms of taste, consistency and dosage.

2. Preparation method according to claim 1, characterised in that the powder obtained at the end of the grinding step has a particle size ranging from 60 to 400 microns, and preferably from 100 to 300 microns.

3. Preparation method according to claim 1 or claim 2, characterised in that the excipient(s) enabling the powder obtained at the end of the grinding step to be transformed into a formulation having a consistency suitable for an administration by oral route such as a gel, a liquid or a paste are chosen among the group constituted by resins and polymers soluble or not soluble in water.

4. Preparation method according to any of claims 1 to 3, characterised in that the excipient(s) enabling the taste of the solid pharmaceutical formulation intended to be ground to be masked are chosen among the group constituted by taste enhancers and flavours.

5. Preparation method according to any of claims 1 to 4, characterised in that, when the quantity of active ingredient in the solid pharmaceutical formulation before grinding varies from 1% to 20% in weight compared to the total weight of the tablet, then the quantity of active ingredient in the final oral formulation varies from 0.1% to 5% in weight compared to the total weight of the formulation.

6. Pharmaceutical formulation suitable for an administration by oral route in terms of taste, consistency and dosage, capable of being obtained according to the method of any of claims 1 to 5.

7. Device or apparatus intended for the preparation of a pharmaceutical formulation suitable for an administration by oral route in terms of taste, consistency and dosage according to claim 6, characterised in that it comprises:
an electric motor (2),
a grinder (6) situated around an axle (5),
a mixer (7) situated around the axle (5),
a compartment (8) enabling a solid formulation to be conveyed up to the grinder (6).

8. Device or apparatus according to claim 7, characterised in that it further comprises:
one or several batteries (1),
one or several pinions (3) and (4),
one or several springs (9) inside the compartment (8),
one or several screws (10) attached to the axle (5) and situated inside the mixer (7).

9. Device or apparatus according to claim 7 or claim 8, characterised in that the electric motor (2) generates the rotation of the pinions (3) and (4), which themselves generate the rotation of the axle (5) and the grinder (6) integral with said axle (5).

10. Device or apparatus according to any of claims 7 to 9, characterised in that the spring(s) (9) of the compartment (8) convey a solid formulation up to the grinder (6).

11. Device or apparatus according to any of claims 7 to 10, characterised in that the mixer (7) and the compartment (8) are detachable parts.

12. Device or apparatus according to any of claims 7 to 11, characterised in that it comprises a syringe enabling dosing comprising:
a main transparent body,
a volume scale,
a detachable cover,
a tapering and sealable outlet.

13. Device or apparatus according to any of claims 7 to 12, characterised in that it is assembled in a leak tight envelope and is equipped with a joint assuring leak tightness between the mixer and the other elements.

14. Device or apparatus intended for the preparation of a pharmaceutical formulation suitable for an administration by oral route in terms of taste, consistency and dosage according to claim 6, characterised in that it comprises:
a syringe in transparent material, equipped with a graduation;
a cover;
a tapering and sealable outlet.

15. Preparation method according to any of claims 1 to 5, characterized in that it comprises the use of a device or apparatus according to any of claims 7 to 14.
